(19) United States
(12) Patent Application Publication
(10) Pub. No.: US 2014/0121468 A1
(43) Pub. Date: May 1, 2014

(54) SPECTROSCOPIC ILLUMINATION DEVICE USING WHITE LIGHT LEDS

(71) Applicant: Halma Holdings, Inc., Cincinnati, OH (US)

(72) Inventor: Jason M. Eichenholz, Orlando, FL (US)

(73) Assignee: Halma Holdings, Inc., Cincinnati, OH (US)

(21) Appl. No.: 14/064,262

(22) Filed: Oct. 28, 2013

Related U.S. Application Data
(60) Provisional application No. 61/718,885, filed on Oct. 26, 2012.

(51) Int. Cl. A61B 1/06

(52) U.S. Cl. CPC A61B 1/0638
USPC 600/249

(57) ABSTRACT

An apparatus for a spectroscopic illumination device for medical diagnosis may include a mount having a proximal end, a distal end, a first electrical connection disposed at the proximal end, a second electrical connection disposed at the proximal end, a light path along a longitudinal axis of the mount, and a lens disposed along the light path at the distal end. The spectroscopic illumination device may also include one or more light emitting diodes coupled within the mount along the light path, each of the one or more light emitting diodes is coated with a phosphoric composition of red, blue, and green phosphors such that the one or more light emitting diodes emits a white light having a color rendering index of greater than about 95, wherein the one or more light emitting diodes are electrically coupled to the first electrical connection and the second electrical connection. The spectroscopic illumination device may also include an optical collection device disposed at the distal end such that an end of the optical collection device is operable to receive a reflected light.

### CROSS REFERENCE TO RELATED APPLICATIONS

The present application hereby claims priority under 35 U.S.C. §119(e) to Provisional U.S. Application No. 61/718,885 filed Oct. 26, 2012, titled “SPECTROSCOPIC ILLUMINATION DEVICES AND METHODS OF USING THE SAME.”

### TECHNICAL FIELD

The present disclosure relates to medical illumination apparatuses and more particularly to medical diagnostic illumination or medical examination illumination using light emitting diodes (LEDs) that mimic natural light.

### SUMMARY

In one embodiment, an apparatus for a spectroscopic illumination device for medical diagnosis may include a mount having a proximal end, a distal end, a first electrical connection disposed at the proximal end, a second electrical connection disposed at the proximal end, a light path along a longitudinal axis of the mount, and a lens disposed along the light path at the distal end. The spectroscopic illumination device may also include one or more light emitting diodes coupled to the mount along the light path, each of the one or more light emitting diodes is coated with a phosphoric composition of red, blue, and green phosphors such that the one or more light emitting diodes emits a white light having a color rendering index of greater than about 95, wherein the one or more light emitting diodes are electrically coupled to the first electrical connection and the second electrical connection. The spectroscopic illumination device may also include an optical collection device disposed at the distal end such that the optical collection device is operable to receive a reflected light.

In another embodiment, an apparatus for a medical evaluation kit for medical evaluation of a patient may include a spectroscopic illumination device including a mount having a proximal end, a distal end, a first electrical connection disposed at the proximal end, a second electrical connection disposed at the proximal end, a light path along a longitudinal axis of the mount, and a lens disposed along the light path at the distal end. The spectroscopic illumination device may also include one or more light emitting diodes coupled to the mount along the light path, each of the one or more light emitting diodes is coated with a phosphoric composition of red, blue, and green phosphors such that the one or more light emitting diodes emits a white light having a color rendering index of greater than about 95, wherein the one or more light emitting diodes are electrically coupled to the first electrical connection and the second electrical connection, and an optical collection device disposed at the distal end such that the optical collection device is operable to receive reflected light from a target. The medical evaluation kit may also include a monitor coupled to the spectroscopic illumination device to display the reflected light and provide power for and adjust an intensity of the one or more light emitting diodes.

In yet another embodiment, an apparatus for a medical illumination system may include a spectroscopic illumination device including a mount having a proximal end, a distal end, a first fiber optic cable at the proximal end, a second fiber optic cable at the proximal end, a light path along a longitudinal axis of the mount, and a lens at the distal end and along the light path, and wherein the mount is formed from FDA Class IV heat shrinkable tubing surrounding medical grade Tygon™ tubing. The spectroscopic illumination device may also include a light source that emits a white light and is along the light path, an optical collection device to capture a reflected white light, a first wavelength selection device along the light path, and a second wavelength selection device along the light path wherein the white light travels along the light path from the light source, passes through the first wavelength selection device, illuminates a target that emits the reflected white light, the reflected white light passes through the second wavelength selection device, and is collected by the optical collection device. The medical illumination system may also include one or more light emitting diodes optically coupled to the spectroscopic illumination device that produce the white light, each of the one or more light emitting diodes is coated with a phosphoric composition of red, blue, and green phosphors to define a color rendering index of greater than 95 and a spectrometer coupled to the spectroscopic illumination device to display the reflected white light and comprising a power source electrically coupled to the one or more light emitting diodes to power and adjust an intensity of the one or more light emitting diodes.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts a schematic representation of a spectroscopy system according to one or more embodiments herein;

FIG. 2 depicts a side view of a catheter probe that incorporates a spectroscopic illumination device according to one or more embodiments herein;

FIG. 3 depicts a schematic representation of another embodiment of the spectroscopy system according to one or more embodiments herein;

FIG. 4 depicts a side view of another embodiment of a catheter probe according to one or more embodiments herein;

FIG. 5 depicts schematic representation of one or more secondary wavelength selection devices along a light path according to one or more embodiments herein;

FIG. 6 depicts schematic representation of one or more primary wavelength selection devices along the light path according to one or more embodiments shown and described herein;

FIG. 7 depicts schematic representation of a combination of one or more primary wavelength selection devices and one or more secondary wavelength selection devices according to one or more embodiments shown and described herein;

FIG. 8 depicts a spectral distribution of the one or more LEDs according to one or more embodiments shown and described herein;

FIG. 9A depicts a CRI of a typical white light LED according to one or more embodiments shown and described herein; and

FIG. 9B depicts the CRI of one or more LEDS of the spectroscopic illumination device according to one or more embodiments shown and described herein.

### DETAILED DESCRIPTION

LEDs are semiconductor light emitters often used as a replacement for other light sources, such as incandescent lamps. They are particularly useful as display lights, warning lights and indicating lights or in other applications where colored light is desired. The color of light produced by an LED is dependent on the type of semiconductor material used in its manufacture and a phosphor blend coating the lens of the LED to produce the desired color of light. For example, to produce a traditional white LED, a blue LED (of about 450 nm in wavelength made from Indium gallium nitride (InGaN)) is doped with cerium doped yttrium aluminum garnet Y₃Al₅0₁₂:Ce³⁺ (“YAG”) to produce a white light. The combination of the blue LED and the doped YAG transforms a fraction of the light produced from the blue LED from a short wavelength (e.g. blue) to longer wavelength (e.g. red) (i.e. the Stokes shift). The spectral distribution of this LED has several peaks and valleys and is not flat across the visible spectrum. The deficiencies in the visible spectrum do not accurately portray certain colors of the illuminated object.

Embodiments shown and described herein include a spectroscopic illumination device that includes a light source that emits a light and may be used in medical diagnostic or medical examination procedures. The emitted light has a spectral distribution that mimics natural light, e.g., white light. The light source may include one or more light sources such as, for example, LEDs pumped with electromagnetic radiation generally within and/or near the ultraviolet (UV) range of the electromagnetic spectrum. The LEDs from the spectroscopic illumination device are used to illuminate a target and allow the spectroscopic illumination device to capture a reflected light from the target which approximates, is substantially similar to or equates to the natural color rendering of the target. Various embodiments of the spectroscopic illumination device will be described in more detail herein.

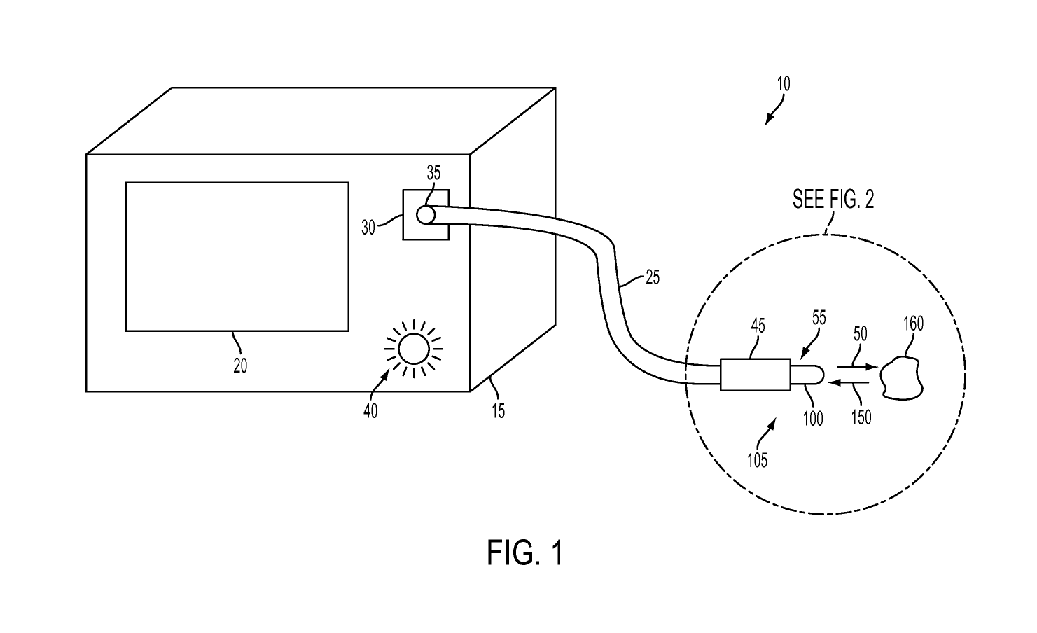

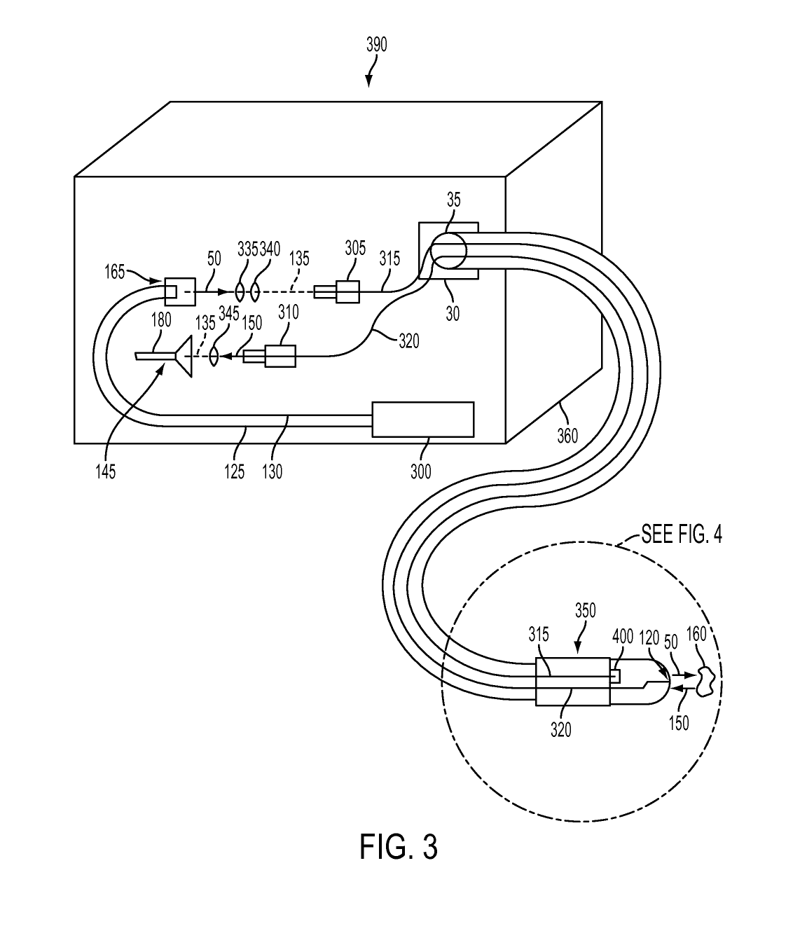

Referring now to FIG. 1, a spectroscopy system 10 is shown. The spectroscopy system 10 may include a monitor 15 and a catheter probe 105. The monitor 15 may house a display 20, a socket 30, an intensity control knob 40, a power supply (as shown in FIG. 3) and other internal components such as electronics which would enable the spectroscopy system 10 to work and will be described in greater detail below. The display 20 may be used to display information pertaining to a spectroscopic examination and/or receive input from a user of the spectroscopy system 10 with relation to the spectroscopic examination. The socket 30 may contain mating connectors that interface with one or more electrical connectors of the catheter probe 105 and/or one or more optical connectors of the catheter probe 105.

The catheter probe 105 may include a spectroscopic illumination device 100. The spectroscopic illumination device 100 may provide illumination that is useful for the spectroscopy system 10 to perform medical diagnosis or medical examination procedures. The spectroscopic illumination device 100 may produce an emitted light 50 that exits the catheter probe 105 and illuminates a target 160. The intensity control knob 40 may be electrically coupled to the power supply and control an intensity of the emitted light 50 by varying a power output of the power supply. Not to be bounded by theory, a portion of the emitted light 50 that reaches the target 160 may be scattered and/or reflected back to the spectroscopic illumination device 100. At least a portion of this scattered and/or reflected light may be received by the spectroscopic illumination device 100 from the target 160 (as used herein after “a reflected light 150”). The reflected light 150 may be diffused or incoherent light or the reflected light 150 may be a focused light. The spectral distribution of the reflected light 150 may be different from the spectral distribution of the emitted light 50 because of the spectral distribution of the reflected light 150 includes color characteristics of the target 160.

The catheter probe 105 may include a plug 35. The plug 35 may coupled with the socket 30 which may include electrical coupling and/or optical coupling between the catheter probe 105 and the monitor 15. Further, a body 45 of the catheter probe 105 may be formed from FDA Class VI heat shrinkable tubing surrounding medical grade Tygon™ tubing. The body 45 may house the spectroscopic illumination device 100 or the spectroscopic illumination device 100 may be coupled to an examination end 55 of the body 45.

Besides the catheter probe 105, the spectroscopic illumination device 100 may also be incorporated into or with a targetable injection needle probe, a catheter probe with an extendable needle, and/or any other type of medical probe suitable for emitting and receiving electromagnetic radiation (e.g. visible light). In another embodiment, the spectroscopic illumination device 100 may not be incorporated into a probe at all. In such an embodiment, the spectroscopic illumination device 100 may be a standalone device in and of itself that may be electrically and/or optically coupled to the monitor 15.

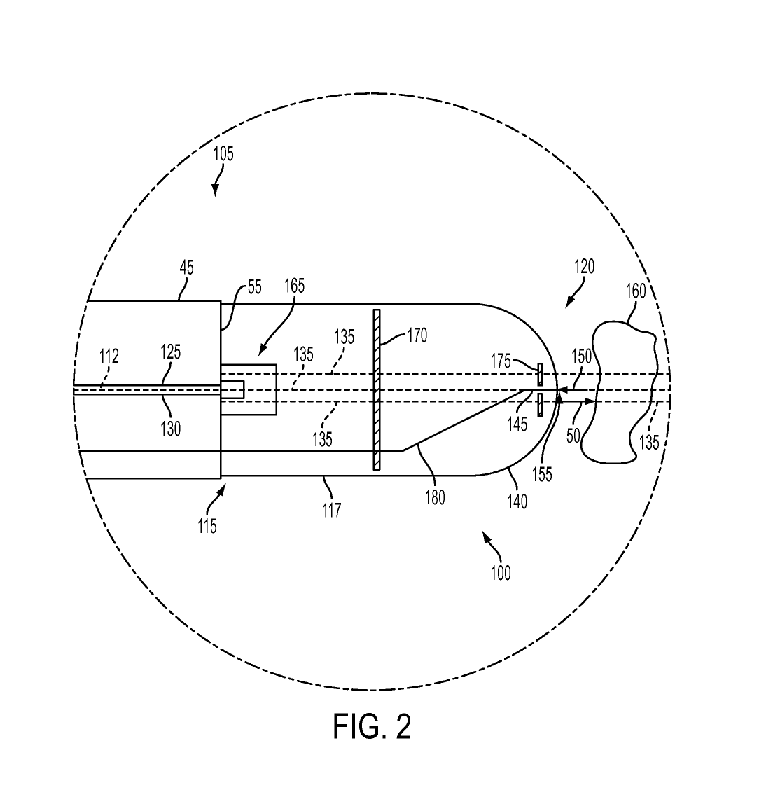

Referring to FIG. 2, a side view of the catheter probe 105 that incorporates the spectroscopic illumination 100 device is shown. The spectroscopic illumination device 100 may include, a mount 110 having a proximal end 115 and a distal end 120, a first electrical connection 125 disposed at the proximal end 115, a second electrical connection 130 disposed at the proximal end 115, a longitudinal axis 112, a light path 135 that lies along and is coaxial with the longitudinal axis 112, a lens 140 disposed along the light path 135 at the distal end 120, and an optical collection device 145 positioned within the mount 110 such that an end 155 of the optical collection device 145 is disposed adjacent to and/or along the light path 135. The first electrical connection 125 and the second electrical connection 130 may be used to facilitate power for the spectroscopic illumination device 100. Although the light path 135 is shown as coaxial with the longitudinal axis 112, it is understood that the light path 135 may have a variety of different pathways, directions, and axes. Objects along the light path 135 are aligned with one another optically. In some embodiments, the light path 135 may include a path that the emitted light 50 and the reflected light 150 traverse. In other embodiments, the light path 135 includes only a path that the emitted light 50 traverses or, in the alternative, only a path that the reflected light 150 traverses. In one embodiment, the catheter probe 150 may include an optional chip (not shown) that retains information useful in operation of the device, such as calibration parameters, a reference database, etc.

The mount 110 may include a housing 117 that is may be made from a plastic, an epoxy, a metal, an insulating sheathing, glass, composite materials, or combinations thereof. The housing 117 may extend from proximal end 115 to the distal end 120 of the mount 110. As set forth above, the lens 140 is connected to or affixed to the distal end 120 along the light path 135. The lens 140 may be a shaped lens and/or configured to change the optical characteristics of the emitted light 50 exiting and/or the reflected light 150 entering the spectroscopic illumination device 100. The lens 140 may include, but not be limited to, a collimating lens, a convex lens, a concave lens, a fresnel lens, a microscope objective lens, butt coupling of the optical collection device 145 against the lens 140, butt coupling of the optical collection device 145 against the target 160, combinations thereof, or any other type of lens capable of providing desired optical characteristic of either the emitted light 50 or reflected light 150.

In another embodiment, the spectroscopic illumination device does not include a lens. In this embodiment, the housing may be configured such that it defines an aperture (not shown) at the distal end 120 sized sufficiently to permit the emitted light 50 to exit the device 100 and the reflected light to be received by the end 155 of the optical collection device 145. In one example, the housing 117 may extend to the distal end 120 in any shape or configuration such as, for example, to form a shaped distal end 120, including but not limited to a conical-shaped end, a spherical-shaped end, etc. In another example, the housing 117 may terminate at the distal end 120 such that the distal end 120 is substantially aligned with and/or co-planar with the end 155 of the optical collection device 145. In this embodiment the distal end is substantially planar in shape. In other embodiments, the distal end 120 may include an angled planar shape. In yet another example, a lens, similar to lens 140, may be connected and/or affixed to the planar or shaped distal end over or within the aperture.

In still yet another example, a protective cover may be disposed over the distal end 130 of any of the aforementioned embodiments to protect the distal end 120, the optical collection device 145, and/or the lens 140. This protective cover may include any variety of shapes, sizes, and/or configurations and be made of any number of materials such as, for example, plastic, glass, metal, aluminum, composites, or combinations thereof. Additionally, at least a portion of the protective cover may be transparent to all or a portion of the emitted light 50 and/or the reflected light 150.

In the embodiment shown in FIG. 2, the optical collection device 145 may be a configured and positioned within the mount 110 to collect the reflected light 150 from the target 160 through the lens 140. In one example, the lens 140 may collimate the reflected light 150 before it is received by the optical collection device 145. In one embodiment, the optical collection device 145 may be black-coated such that the optical collection device 145 is shielded from stray light from the one or more LEDs 165. As an example, the one or more LEDs 165 emit light that travels along the light path 135 within the spectroscopic illumination device 100 and through the lens 140, exiting the device 100 as emitted light 50. A portion and/or all of the emitted light 50 illuminates a target 160 at which point a portion of this light is scattered and/or reflected by the target 160 back towards the spectroscopic illumination device 100, i.e., the reflected light 150. A portion and/or all of the reflected light 150 may be received by the optical collection device 145, which may then be transmitted to the monitor 15 of FIG. 1.

The spectroscopic illumination device 100 may also include one or more LEDs 165 coupled to the mount 110 along the light path 135. The one or more LEDs 165 are electrically coupled to the first electrical connection 125 and the second electrical connection 130. The one or more LEDs 165 may act as a light source for the spectroscopic illumination device 100 and for a variety of spectroscopic equipment, instruments, and/or spectroscopic medical diagnostic and/or examination procedures. The one or more LEDs 165 may be embedded in the mount 110 or the one or more LEDs 165 may otherwise be incorporated into the examination end 55 of the catheter probe 105.

The first electrical connection 125 and the second electrical connection 130 may be coupled to the power supply (FIG. 3) in the monitor 15. The power supply may provide the power needed to operate the spectroscopic illumination device 100. In another embodiment, a portable power source disposed within or along the catheter probe 105 may provide power for the one or more LEDs 165. In this embodiment, the first electrical connection 125 and the second electrical connection 130 may be coupled to portable power source. The portable power source may comprise one or more battery cells, a capacitor, a solar cell, or the like. In such embodiments, the plug 35 and socket 30 may lack electrical connectors to couple the plug 35 to the socket 30 as power from the power supply in the monitor 15 may not be needed. The power to operate the spectroscopic illumination device 100 may be completely provided by the portable power source within or along the catheter probe 105.

The one or more LEDs 165 may be arranged in an array. The array may comprise one or more clusters. Each cluster may be a linear cluster, a staggered cluster, a herringbone cluster, a honeycomb cluster, a triangular cluster, a hexagonal cluster, a circular cluster, or combinations thereof.

By way of an example, the one or more LEDs 165 may act as a spectroscopy illuminator in any of the spectroscopy systems shown and described in U.S. Pat. No. 6,711,426, which is herein incorporated by reference in its entirety. As another example, the one or more LEDs 165 may act as a light source in a spectrometer such as those commercially available from Spectros, Inc. of the United States.

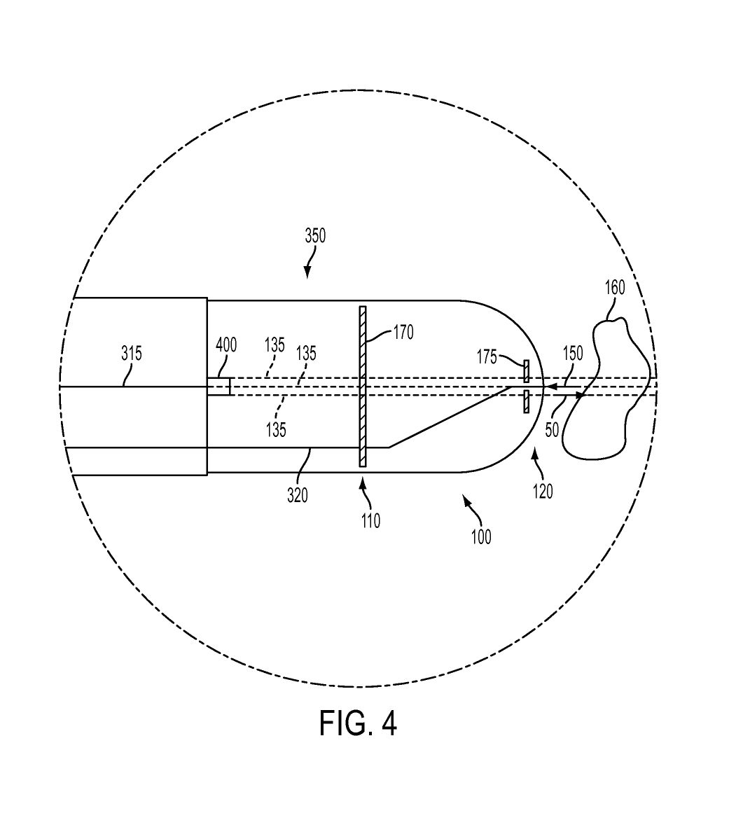

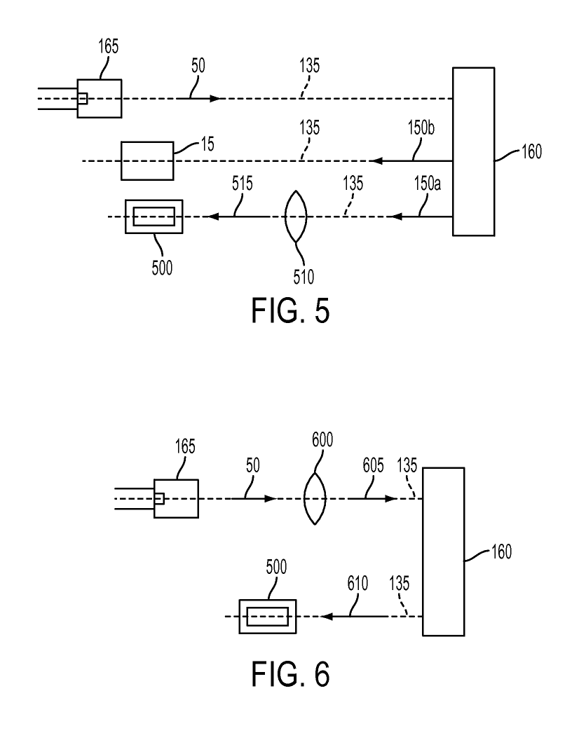

The spectroscopic illumination device 100 may also include one or more wavelength selection devices along the light path 135. As an example, spectroscopic illumination device 100 may comprise a first wavelength selection device 170, a second wavelength selection device 175, or both as shown, for example, in FIGS. 2 and 4. The one or more wavelength selection devices may be placed along the light path 135 to select or exclude frequencies based upon a selection characteristic of the wavelength selection device. The one or more wavelength selection devices are described in greater detail below in FIGS. 5, 6, and 7.

Referring to FIGS. 1 and 2, the catheter probe 105 may be either coupled to the monitor 15 or the catheter probe 105 may be electrically coupled and/or optically coupled to the monitor 15 via a connector cable 25. The connector cable 25 may include the plug 30 which couples with the socket 35 to provide electrical coupling and/or optical coupling between the catheter probe 105 and the monitor 15. The electrical coupling of the catheter probe 105 to the monitor 15 may include the transmission of power and/or data between the catheter probe 105 and the monitor 15 through the one or more electrical connectors (not shown). The optical coupling of the catheter probe 105 to the monitor 15 may include transmission of light and thus, for example, data, between the catheter probe 105 and the monitor 15 through the one or more optical connectors (not shown).

For example, still referring to FIGS. 1 and 2, in one embodiment, the optical collection device 145 may be optically coupled to an image data feed 180. The image data feed 180 may be an optical fiber. In this embodiment, the reflected light 150 may be collimated through a collimating lens (not shown) and transmitted to the monitor 15 via the optical fiber. This collimating lens may be lens 140 or may be a separate and distinct lens from lens 140. In another embodiment, the optical collection device 145 may be electrically coupled to the image data feed 180. The image data feed 180 may be an electrical wire, pair of electrical wires, a CAT cable, a coaxial cable, a twisted pair cable, a shielded cable, or the like. In this embodiment, the optical collection 145 device may be a camera (not shown), image sensor, other electronic device configured to capture images. The reflected light 150 may be captured by the optical collection device 145 and converted to an image data (e.g. captured image) and may be transmitted to the monitor 15 via the image data feed 180. Examples of the camera and/or image sensor of this embodiment may include a charge-coupled device (“CCD”) sensor and/or a complimentary metal-oxide semiconductor (“CMOS”) sensor. Illustrative CCD and CMOS devices are shown and described in the one or more of the following U.S. Pat. Nos. 5,436,492; 5,844,264; 5,894,943; or 7,414,655; 7,336,757 and/or 3,356,858; 5,108,938; 6,084,229; 6,909,591; or 7,595,660, which are herein incorporated by reference in their entirety.

Referring to FIGS. 3 and 4, another embodiment of a spectroscopy system 390 comprising a monitor 360 and a catheter probe 350. This embodiment includes the one or more LEDs 165 and the optical collection device 145 housed in the monitor 360, rather than housed in the catheter probe 105 as described above with reference to the embodiment in FIG. 2. The power supply 300 may be electrically coupled to the one or more LEDs 165 via the first electrical connection 125 and the second electrical connection 130. The one or more LEDs 165 may be optically aligned along the light path 135. The emitted light 50 from the one or more LEDs 165 travels along the light path 135 and passes through a collimating lens 335. The collimating lens 335 may be configured to collimate the emitted light 50 into a first optical socket 305. A third wavelength selection device 340 of the one or more wavelength selection devices may be positioned along the light path 135 between the one or more LEDs 165 and the first optical socket 305. The third wavelength selection device 340 is an optional device and some embodiments may eliminate it from monitor 360. The first optical socket 305 may be optically coupled to a first optical fiber 315. The first optical socket 305 may optically collect the emitted light 50 along the light path 135 and optically transmit the emitted light 50 through the socket 30 and plug 35 via one or more optical connectors. The first optical fiber 315 may be coupled to an emitter 400 in the catheter probe 350.

Referring specifically to FIG. 4, the first optical fiber 315 is optically connected to the emitter 400. The emitter 400 is disposed within and/or coupled to the mount 110 and emits the emitted light 50 along the light path 135. The emitted light 50 travels along the light path 135 through the spectroscopic illumination device 100, exits the distal end 120 of the mount 110, and illuminates the target 160. The reflected light 150 is received by a second optical fiber 320 along the light path 135. The second optical fiber 320 may be coupled to the catheter probe 350 at the distal end 120 and configured to capture the reflected light 150 from the target 160. In some embodiments, a collimating lens (not shown) is used to collimate the reflected light 150 into the second optical fiber 320. The first wavelength selection device 170 and the second wavelength selection device 175 may be used in the spectroscopic illumination device 100 and may operate as described above with reference to FIG. 2.

Referring to FIGS. 3 and 4, the second optical fiber optically transmits the reflected light 150 from the catheter probe 350 to a second optical socket 310. The second optical socket 310 may transmit the reflected light 150 along the light path 135 to the optical collection device 145. A fourth wavelength selection device 345 of the one or more wavelength selection devices may be positioned along the light path 135 between the second optical socket 310 and the optical collection device 145. The fourth wavelength selection device 345 is an optional device and some embodiments may eliminate it from the monitor 360. The optical collection device 145 is coupled to the image data feed 180 which is electrically and/or optically coupled to the monitor 360.

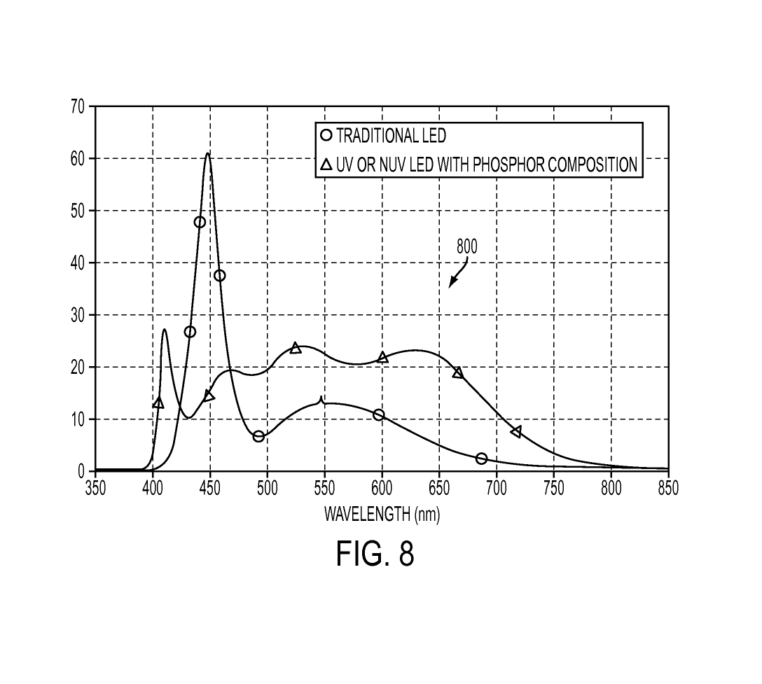

Referring to FIGS. 1 and 3, the spectroscopy systems 10 and/or 390, respectively, may incorporate a spectrometer in lieu of the monitor 15/360. The spectrometer may be coupled to the spectroscopic illumination device 100 and may display the spectral distribution 800 of the reflected light 150 as shown in FIG. 8. The components of the monitors 15 and 360 shown in FIGS. 1 and 3, respectively, are applicable to the spectrometer.

FIGS. 5, 6, and 7 depicts the positioning of one or more wavelength selection devices along the light path 135. Illustrative one or more wavelength selective devices may include, but are not limited to, polarization filters, dichroic filters, dichroic mirrors, dichroic reflectors, reflective filters, thin film filters, interference filters, gel film filters, band pass filters, interference bandpass filters (e.g., exciter filters, barrier filters, etc.), any other optical, color filtering, or interference devices, or combinations thereof. The one or more wavelength selective devices are may be a very accurate color filter used to selectively pass light of certain wavelengths (e.g., visible light), while reflecting or absorbing light of other wavelengths (e.g., UV and/or NUV light). For example, the one or more wavelength selection devices may be used to protect a human eye from UV and/or NUV light when observing reflected light 150 from the target 160. Additionally, the one or more wavelength selective devices may be a part of, optically connected to, or used with the spectroscopic illumination device 100 described herein. As stated above, the one or more wavelength selection devices may optionally be used in the spectroscopy system 10 of FIG. 1 or the spectroscopy system 390 of FIG. 3. Methods to make such wavelength selective devices are shown and described in one or more of the following U.S. Pat. Nos. 5,711,889; 6,638,668; 6,700,690; 7,149,033; 7,648,808 and the following pending U.S. pending application Ser. Nos. 11/511,551 and 12/321,838, which are all herein incorporated by reference in their entirety.

Referring to FIG. 5, one or more secondary wavelength selection devices 510 are shown along at least a portion of the light path 135. The one or more LEDs 165 of one or more illustrative spectroscopy systems shown and described herein emit the emitted light 50 along the light path 135 to illuminate the target 160. As shown at least a first portion 150a of the reflected light from the target 160 passes through one or more secondary wavelength selection devices 510. The one or more secondary wavelength selection devices 510 may be operable to change the light characteristics of the first portion 150a of the reflected light to create a filtered reflected light 515 received by an imager 500. The imager 500 may be a human eye and/or the optical collection device 145 as shown and described above herein. The second wavelength selection device 175 of FIGS. 2 and 4 and/or the fourth wavelength selection device 345 of FIG. 3 may be examples of the one of the one or more secondary wavelength selection devices 510.

As also shown, at least a second portion 150b of the reflected light 150 may bypass the one or more secondary wavelength selection devices 510 and be received in an unfiltered state by to the monitor 15. Although the monitor 15 is shown, it is understood that the monitor 360 can be interchanged with the monitor 15.

To further illustrate this point, the target 160 may be illuminated with UV frequencies and/or NUV frequencies from the one or more LEDs 165. If the imager 500 is a human eye and the one or more LEDs 165 are UV and/or NUV LEDs, the imager 500 (e.g. human eye) may be exposed to damaging UV and/or NUV frequencies. In this example, the imager 500 will require that the UV and/or NUV frequencies be removed from the reflected light 150 before reaching the imager 500. Thus, the first portion 150a of reflected light that would otherwise be received by the imager 500 is filtered by the one or more secondary wavelength selection devices 510 to remove or block UV and/or NUV frequencies from the first portion 150b of reflected light. Continuing the example, the second portion 150b of reflected light may be transmitted to the monitor 15 without any filtering of the reflected light 150 by the one or more secondary wavelength selection devices 510 to enable the monitor 15 (or spectrometer) to perform spectral analysis on the unfiltered reflected light.

FIG. 6 depicts one or more primary wavelength selection devices 600 along the light path 135. The one or more LEDs 165 of one or more illustrative spectroscopy systems shown and described herein emit the emitted light 50 along the light path 135. The emitted light 50 passes through the one or more primary selection devices 600 along the light path 135. The one or more primary selection devices may be operable to change the light characteristics of the emitted light 50 to create a filtered emitted light 605. The filtered emitted light 605 illuminates the target 160 along the light path 135. A primary reflected light 610 includes only those frequencies of light allowed by the one or more primary wavelength selection devices 600. The first wavelength selection device 170 of FIG. 2, the second wavelength selection device 175 of FIG. 2, and/or the third wavelength selection device 340 of FIG. 3 may be examples of the one or more primary wavelength selection devices 600.

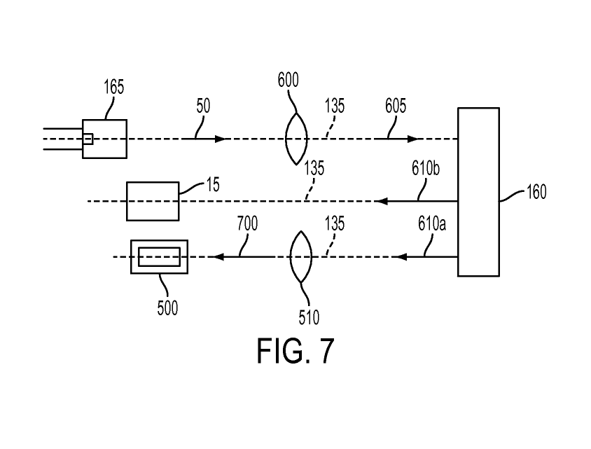

FIG. 7 depicts the combination of one or more primary wavelength selection devices 600 and one or more secondary wavelength selection devices 510. The one or more LEDs 165 of one or more illustrative spectroscopy systems shown and described herein emit the emitted light 50 along the light path 135. The emitted light 50 passes through the one or more primary selection devices 600 along the light path 135. The filtered emitted light 605 illuminates the target 160 along the light path 135. At least a first portion 610a of the primary reflected light 610 passes through the one or more secondary wavelength selection devices 510. At least a second portion 610b of the primary reflected light 610 may be collected and transmitted to the monitor 15 (or monitor 360 of FIG. 3) for separate analysis without passing through or being filtered by the one or more secondary wavelength selection devices 510. The one or more secondary wavelength selection devices 510 change the light characteristics of the primary reflected light 610 to create a secondary reflected light 700 received by the imager 500.

The spectral distribution of a LED affects how an object's surface that is illuminated by the LED is seen. Spectral distribution provides a color rendering capability measured by the color rendering index (CRI). Color rendering is defined as the effect of an illuminant (light source) on the color appearance of objects by conscious or subconscious comparison with their color appearance under a reference illuminant. Therefore, the CRI is a measure of how a light source reproduces the colors of an object when compared to how an ideal or natural light source reproduces the colors of the same object. The CRI is commonly defined as a mean R-value for 8 standard color samples (R₁₋₈), usually referred to as the General Color Rendering Index and abbreviated as Ra. In other words, Ra is the average of the R-values for R₁ through R₈. A CRI of 100 indicates that the light source color rendering ability is the same as natural light. The CRI also includes six other specific colors indices (R₉-R₁₄) that are not part of the Rₐ average of R₁ through R₈.

When CRI is measured, the light source is tested against the first 8 pigment color samples, e.g. R₁ through R₈. The remaining 6 represent 4 saturated solids (R₉-R₁₂) and 2 earth tones (R₁₃ and R₁₄). The current CRI scale does not cover the strong reds that are prevalent in skin tones and represented by the red saturated solid, R₉. Therefore, R₉-R₁₄ are given as separate R-values along with the CRI of the light source.

The LEDs of the present disclosure, when configured to have a good spectral distribution (high CRI), may illuminate an object without changing, or with only minimal change of, its natural colors. Depending on the color of the LED and the phosphors used to coat that LED, a variety of different colors can be employed, each with a differing CRI. If several phosphor layers of distinct colors are applied to a LED, the emitted spectrum is broadened, effectively raising the CRI of that given LED to about the high 80s to low 90s. For example, the color red may be seen from a red blood cell illuminated by natural light as opposed to a pinkish color seen from a red blood cell illuminated by a typical white LED. In particular, the R-value R₉, which indicates the color rendering for the strong red, is very important for a range of applications, especially of a medical nature. Therefore, a typical LED with a low R₉ produces poor color rendering of an organic object such as a blood cell which makes the typical white LED unusable for medical applications.

For example, some medical institutions are required to comply with the Cyanosis Observation Index (COI) that imparts strict guidelines for the use of lamps in the diagnosis of patients in hospitals wards, medical clinics, and associated areas. The COI is important to observe a bluish discoloration in the skin and mucous membranes that may indicate that the oxygen levels in the blood are depleted. The condition may go unnoticed by a practitioner trying to diagnose a patient if the red frequencies (660 nm) of the light source used to aid the practitioner are deficient. The visual detection of cyanosis is related to the differences in the spectral transmission of oxyhaemoglobin and reduced heamoglobin which is maximized at about 660 nm. If the light source 660 nm output is too low, the practitioner may diagnose cyanosis when it doesn't exist. If the light source 660 nm output is too high, the light from the light source may mask the cyanosis condition. Therefore, the R₉ R-value is an important color index for medical instruments.

Referring to FIGS. 1-7, each of the one or more LEDs 165 may be coated with a phosphoric composition such that the one or more LEDs 165 emits a white light having a CRI of greater than about 95. Further, the white light may have a R₉ value that is about 97 or greater. As used herein, the terms “phosphor” and “phosphor material” may be used to denote both a single phosphor as well as a composition of two or more phosphors. In one embodiment, the one or more LEDs 165 may comprise a wavelength of about 200 nm to about 440 nm. In another embodiment, the one or more LEDs 165 may comprise a wavelength of about 200 nm to about 440 nm.

Still referring to FIGS. 1-7, in one embodiment, the one or more LEDs 165 may comprise one or more ultraviolet (UV) and/or near-ultraviolet (NUV) pumped LEDs (e.g., LEDs emitting electromagnetic radiation having wavelengths from about 300 nm to about 425 nm, particularly from about 370 nm to about 410 nm, more particularly about 405 nm). As used herein, “pumped” or “pumping” is a process in which light is used to raise (or “pump”) electrons from a lower energy level in an atom or molecule to a higher one. A UV pumped LED and/or a NUV pumped LED has a broader, flatter, spectral response than a non-pumped LED. The term spectral response describes a spectral distribution of light and is elaborated on in greater detail below. The broader and flatter the spectral distribution of light, or spectral response, of a light source, the closer the emitted light of a light source is to mimicking natural white light. In another embodiment, the one or more LEDs may comprise one or more UV and/or NUV pumped LEDs, each one or more UV and/or NUV pumped LED comprising a phosphor composition of a red phosphor, a green phosphor, a blue phosphor, or combinations thereof coated thereon. The combination of the red phosphor, the green phosphor, and the blue phosphor provide a phosphor composition that is excitable at UV and/or NUV frequencies. The combination of the phosphor composition and the UV and/or NUV frequencies provide a broad, flat spectral response that mimics natural white light (refer to FIG. 8). In yet another embodiment, the one or more LEDs 165 may be a pumped laser that emits the white light that mimics natural white light.

In another embodiment, the one or more LEDs 165 may be coated with one or more of the following phosphors: red phosphor, green phosphor, blue phosphor, yellow phosphor, orange phosphor, violet phosphor, any other color of phosphor, any color mixture therebetween, and/or any combinations thereof. The phosphor types and amounts used in the one or more LEDs 165 may be manipulated (by adding and/or subtracting the phosphor and/or blending different types of phosphors) to optimize the reflected light 150 and hence the contrast of an image produced from the reflected light 150. For example, in one embodiment, the phosphors may be blended and applied to coat the one or more LEDs 165. The phosphors may include a minimum of three spectral constituents, a blue phosphor with a peak wavelength of about 460 nanometers (nm) and a spectral width of about 150 nm, a green phosphor with a peak wavelength of about 560 nm and a spectral width of about 100 nm, and a red phosphor with a peak wavelength of about 670 nm and a spectral width of about 150 nm.

In yet another embodiment, the one or more LEDs 165 may comprise LEDs that are pumped with a blue light rather than UV and/or NUV. In other words, the one or more LEDs 165 may emit the blue light such as, for example, electromagnetic energy having wavelength from about 440 nm to about 490 nm. The phosphor coating on the one or more LEDs 165 for this embodiment may be adjusted to achieve the desired CRI, R₉, CQS, and/or other illumination properties as described in greater detail below.

The phosphors coating the one or more LEDs 165 may also be blended to emit a customized spectrum (i.e., a non-uniform spectrum that may emphasis a particular frequency or a particular range of frequencies). For example, the phosphors coating the one or more LEDs 165 may comprise optimized compositions and percentages for maximum visualization of a retina and provide maximum contrast to an image produced from the reflected light 150.

FIG. 8 illustrates a spectral distribution 800 of the one or more LEDs 165 of the present disclosure designated by the triangle points compared to a traditional white LED (i.e., a blue LED of about 450 nm in wavelength made from Indium gallium nitride (InGaN) doped with cerium doped YAG) designated by the circle points. The spectral distribution 800 of the one or more LEDs 165 (designated by the triangle points) is extremely close to that of natural sunlight, meaning that the one or more LEDs 165 may emit white light that is ideally suited to reproducing the actual or natural colors of the target 160 being illuminated. The one or more LEDs 165 of FIGS. 1-7 may be operable to produce a R-value of about greater than 80, about greater than 90, about greater than 95, greater than about 96, greater than about 97, greater than about 98, greater than about 99, from about 97 to about 99, or about 100. This color high rendering achieves subtle color expressions that were considered impossible with traditional white LEDs (designated by the circle points). Further, certain embodiments of the spectroscopic illumination devices and methods shown and disclosed herein may be operable to produce a NIST Color Quality Scale (CQS) value that is improved over conventional devices and methods and about equal to the CRI of the one or more LEDs 165.

The peaked spectra of the traditional white LED (designated by the circle points) inhibits the performance of the traditional white LED (designated by the circle points) when rendering highly saturated colors because their spectral distribution 800 does not adequately span the range of the visible spectrum. The peak spectra enhances colors in the 450 nm range but poorly renders colors in the 700 nm range. When compared to the spectral distribution 800 of the typical white LED (designated by the circle points), the one or more LEDS 165 of the present disclosure provides a relatively flat spectral distribution 800 that renders the colors of the visible spectrum more evenly between about 400 nm to about 750 nm. The improved rendering of colors across the visible spectrum enables a user of the spectroscopic illumination device 100 to differentiate differing medial ailments based on color.

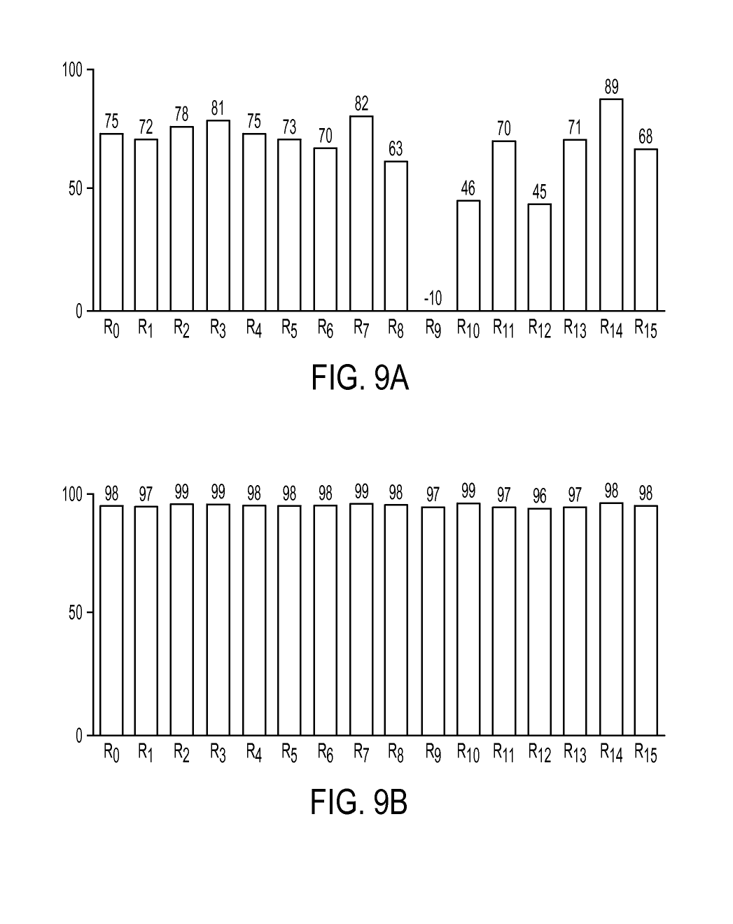

Referring now to FIGS. 9A and 9B, illustrates the improved R9 value of the one or more LEDs 165 of FIG. 2. FIG. 9A depicts a traditional white light LED. FIG. 9B depicts the one or more LEDS 165 of the spectroscopic illumination device 100 of FIG. 2. The vertical axis indicates the R-value for each of the horizontal axis color indices (R₁-R₁₅). Comparing FIG. 9A to FIG. 9B, the R9 value changes from about −10 for a traditional white light LED to about 97 for the one or more LEDs 165. Further, it is noted that the overall color indices for the one or more LEDs 165 is within a small range (97-99) when compared to the color indices for traditional white light LEDs (−10-89).

Illustrative UV and/or NUV LEDs for use as the one or more LEDs 165 are shown and described in U.S. Pat. No. 7,646,032, which is herein incorporated by reference in its entirety. Other illustrative UV and/or NUV LED's that may be used as the one or more LEDs 165 include, but are not limited to, U.S. Pat. Nos. 7,267,787; 7,267,887, and 7,267,719; 7,915,627; and U.S. Patent Publication Nos. 2006/0027781; 2008/0252197; 2008/0073616, which are all herein incorporated by reference in their entirety.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A spectroscopic illumination device for medical diagnosis, comprising:
a mount having a proximal end, a distal end, a first electrical connection disposed at the proximal end, a second electrical connection disposed at the proximal end, a light path along a longitudinal axis of the mount, and a lens disposed along the light path at the distal end;
one or more light emitting diodes coupled to the mount along the light path, each of the one or more light emitting diodes is coated with a phosphoric composition of red, blue, and green phosphors such that the one or more light emitting diodes emits a white light having a color rendering index of greater than about 95, wherein the one or more light emitting diodes are electrically coupled to the first electrical connection and the second electrical connection; and
an optical collection device disposed at the distal end such that the optical collection device is operable to receive a reflected light.

2. The spectroscopic illumination device of claim 1, further comprising one or more wavelength selection devices along the light path.

3. The spectroscopic illumination device of claim 2, wherein at least one of the one or more wavelength selection devices reduces or eliminates an ultraviolet wavelength spectrum and a near-ultraviolet wavelength spectrum from the white light or the reflected light.

4. The spectroscopic illumination device of claim 2, wherein at least one of the one or more wavelength selection devices filters all wavelengths, except a visible spectrum, from the white light or the reflected light.

5. The spectroscopic illumination device of claim 2, wherein a wavelength selection device of the one or more wavelength selection devices is positioned along the light path such that the white light emitted from the one or more light emitting diodes passes through the wavelength selection device.

6. The spectroscopic illumination device of claim 2, wherein a wavelength selection device of the one or more wavelength selection devices is positioned within the mount adjacent the optical collection device such that the reflected light passes through the wavelength selection device prior to being received by the optical collection device.

7. The spectroscopic illumination device of claim 2, wherein the one or more wavelength selection devices comprises a first wavelength selection device along the light path and a second wavelength selection device along the light path.

8. The spectroscopic illumination device of claim 7, wherein the white light emitted from the one or more light emitting diodes passes through the first wavelength selection device, and the reflected light from a target passes through the second wavelength selection device prior to being collected by the optical collection device.

9. The spectroscopic illumination device of claim 2, wherein the one or more wavelength selection devices is a polarizing filter, a dichroic filter, a dichroic mirror, a dichroic reflector, a reflective filter, a thin film filter, an interference filter, a gel film filter, a band pass filter, an interference bandpass filter, a color filter, an interference device, or combinations thereof.

10. The spectroscopic illumination device of claim 1, further comprising a portable power source coupled to the mount at the proximal end and electrically coupled to the first electrical connection and the second electrical connection.

11. The spectroscopic illumination device of claim 1, wherein the optical collection device is a fiber optic cable.

12. The spectroscopic illumination device of claim 1, wherein each of the one or more light emitting diodes is an ultra-violet light emitting diode.

13. The spectroscopic illumination device of claim 1, wherein each of the one or more light emitting diodes is a near ultra-violet diode.

14. The spectroscopic illumination device of claim 1, wherein the one or more light emitting diodes are arranged in an array with one or more clusters.

15. The spectroscopic illumination device of claim 14, wherein each of the one or more clusters is a linear cluster, a staggered cluster, a herringbone cluster, a honeycomb cluster, a triangular cluster, a hexagonal cluster, a circular cluster, or combinations thereof.

16. The spectroscopic illumination device of claim 1, wherein the white light has a R9 value that is about 97.

17. The spectroscopic illumination device of claim 1, wherein the one or more light emitting diodes comprise a wavelength of about 200 nm to about 440 nm.

18. The spectroscopic illumination device of claim 1, wherein the one or more light emitting diodes comprise a wavelength of about 370 nm to about 410 nm.

19. The spectroscopic illumination device of claim 1, wherein:
the blue phosphor has a peak wavelength of about 460 nanometers (nm) and a spectral width of about 150 nm;
the green phosphor has a peak wavelength of about 560 nm and a spectral width of about 100 nm; and
the red phosphor has a peak wavelength of about 670 nm and a spectral width of about 150 nm.

20. A medical evaluation kit for medical evaluation of a patient, comprising:
a spectroscopic illumination device comprising:
a mount having a proximal end, a distal end, a first electrical connection disposed at the proximal end, a second electrical connection disposed at the proximal end, a light path along a longitudinal axis of the mount, and a lens disposed along the light path at the distal end,
one or more light emitting diodes coupled to the mount along the light path, each of the one or more light emitting diodes is coated with a phosphoric composition of red, blue, and green phosphors such that the one or more light emitting diodes emits a white light having a color rendering index of greater than about 95, wherein the one or more light emitting diodes are electrically coupled to the first electrical connection and the second electrical connection, and
an optical collection device disposed at the distal end such that the optical collection device is operable to receive reflected light from a target; and
a monitor coupled to the spectroscopic illumination device to display the reflected light and provide power for and adjust an intensity of the one or more light emitting diodes.

21. The medical evaluation kit of claim 20, further comprising one or more wavelength selection devices along the light path.

22. The medical evaluation kit of claim 20, further comprising a catheter probe coupled between the spectroscopic illumination device and the monitor.

23. The medical evaluation kit of claim 21, wherein the one or more wavelength selection devices comprises a first wavelength selection device that reduces or eliminates an ultraviolet wavelength spectrum and a near-ultraviolet wavelength spectrum from the white light or the reflected light.

24. The medical evaluation kit of claim 20, wherein each of the one or more light emitting diodes is an ultra-violet light emitting diode.

25. The medical evaluation kit of claim 20, wherein each of the one or more light emitting diodes is a near ultra-violet diode.

26. The medical evaluation kit of claim 20, wherein the one or more light emitting diodes are arranged in an array with one or more clusters.

27. The medical evaluation kit of claim 20, wherein:
the blue phosphor has a peak wavelength of about 460 nanometers (nm) and a spectral width of about 150 nm;
the green phosphor has a peak wavelength of about 560 nm and a spectral width of about 100 nm; and
the red phosphor has a peak wavelength of about 670 nm and a spectral width of about 150 nm.

28. A medical illumination system that emits white light for use in medical diagnostic and examination procedures, comprising:
a spectroscopic illumination device comprising:
a mount having a proximal end, a distal end, a first fiber optic cable at the proximal end, a second fiber optic cable at the proximal end, a light path along a longitudinal axis of the mount, and a lens at the distal end and along the light path, and wherein the mount is formed from FDA Class IV heat shrinkable tubing surrounding medical grade Tygon™ tubing,
a light source that emits a white light and is along the light path,
an optical collection device to capture a reflected white light,
a first wavelength selection device along the light path,
a second wavelength selection device along the light path wherein the white light travels along the light path from the light source, passes through the first wavelength selection device, illuminates a target that emits the reflected white light, the reflected white light passes through the second wavelength selection device, and is collected by the optical collection device;
one or more light emitting diodes optically coupled to the spectroscopic illumination device that produce the white light, each of the one or more light emitting diodes is coated with a phosphoric composition of red, blue, and green phosphors to define a color rendering index of greater than 95; and
a spectrometer coupled to the spectroscopic illumination device to display the reflected white light and comprising a power source electrically coupled to the one or more light emitting diodes to power and adjust an intensity of the one or more light emitting diodes.

29. The medical illumination system of claim 28, wherein the one or more light emitting diodes are coupled to the spectrometer.

30. The medical illumination system of claim 28, wherein the one or more light emitting diodes are coupled to the spectroscopic illumination device.

31. The medical illumination system of claim 28, wherein:
the blue phosphor has a peak wavelength of about 460 nanometers (nm) and a spectral width of about 150 nm;
the green phosphor has a peak wavelength of about 560 nm and a spectral width of about 100 nm; and
the red phosphor has a peak wavelength of about 670 nm and a spectral width of about 150 nm.
